(19) United States
(12) Patent Application Publication
(10) Pub. No.: US 2016/0018490 A1
(43) Pub. Date: Jan. 21, 2016

(54) BODY-INSERTABLE DEVICE WITH AN MR COIL AND A SHIN COIL

(71) Applicant: Siemens Aktiengesellschaft, Muenchen (DE)

(72) Inventor: Stephan Biber, Erlangen (DE)

(73) Assignee: Siemens Aktiengesellschaft, Muenchen (DE)

(21) Appl. No.: 14/801,202

(22) Filed: Jul. 16, 2015

(30) Foreign Application Priority Data
Jul. 16, 2014 (DE) 102014213857.4

(51) Int. Cl. G01R 33/34; A61B 5/055; G01R 33/3875

(52) U.S. Cl. CPC G01R 33/34084; G01R 33/3875; A61B 5/055

(57) ABSTRACT

A medical device to be introduced into the body of a subject has at least one MR coil that can be introduced into the body at the same time and at least one shim coil that can be introduced into the body at the same time. The device can be formed as an endorectal probe, vaginal probe and transesophageal probe as well as an implant.

### BACKGROUND OF THE INVENTION

1. Field of the Invention

The invention relates to a device to be introduced at least partially into a body, the device having at least one MR coil that can be introduced into the body at the same time. The invention can be applied in particular to medical probes such as endorectal probes, vaginal probes, and transesophageal probes as well as to implants, etc.

2. Description of the Prior Art

Imaging in magnetic resonance tomography (also abbreviated as MRT or MRI) is based on the spins of atomic nuclei aligned in a B0 basic field. For many applications, the homogeneity of the B0 basic field (i.e. the presence of an identical field strength in a large three-dimensional volume) is of major significance for image quality, as well as for spatial registration of the images. The use of fat saturation methods is important for many imaging technologies. With a fat saturation method, the fatty tissue, which produces a strong signal in many contrast modes, is masked out. Masking out is frequently essential if the MR images are to be used for diagnostic purposes, as in many sequence types pathological tissue shows a similar or the same contrast response to fat. Functional fat saturation is therefore of major significance for many cases.

Various methods are currently used for fat saturation, for example the so-called Dixon method or spectral fat saturation. Spectral fat saturation and associated techniques make use of the fact that fat and water have slightly different resonant frequencies (difference of approx. 3.1 ppm between fat and water). A powerful transmit pulse on the fat frequency can suppress the fat signal, without influencing the imaging of the protons associated with the water molecules. The functionality of all the methods based on the spectral separation of fat and water, however, depends on the homogeneity of the B0 basic field. If the B0 basic field varies to a similar degree to the spectral separation of fat and water (approx. 3.1 ppm), the fat and water resonances can no longer be separated spectrally.

Current superconducting magnets allow magnetic field homogeneities with differences of less than 1 ppm over a volume of approx. 30×40×50 cm. Problems with fat saturation therefore tend to occur in outer regions of the anatomy (e.g. in the region of a shoulder or neck), which also cannot be supported centrally in the core of an MR magnet, due to the lack of space.

Even more critical than the (known and deterministic) inhomogeneities of the B0 basic field are the inhomogeneities introduced by the actual tissue of a patient. Human tissue has a relative magnetic permeability that is different from 1.000000. As a result, discontinuities between air and tissue produce significant B0 distortions. The inhomogeneous distribution of water, air, bone, fat, etc. in the human body also results in another distortion of the B0 basic field that is different for each patient.

When examining tissue within a body, probes are used for many applications. The probes have MR receive coils and/or MR transmit coils and are introduced into the body via an opening in the body. Examples of such are an endorectal probe for a prostate examination, a vaginal probe for a cervical examination, and a transesophageal probe for applications in the mouth, esophagus, stomach/bowel, or a probe for imaging in body openings created during an operation.

FIG. 3 shows a sectional side view of an outline of an MR layout for a prostate examination. A male body K has been introduced into an examination tunnel of an MR scanner (top diagram) for this purpose. The examination tunnel has one PPA (Pelvic Phased Array) MR coil 101 in frontal in relation to the body and one rear PPA MR coil 102. Also introduced into the body K is an endorectal probe 103, which has an expandable device head 104 (also referred to as the endocoil). The device head 104 is moved into the vicinity of the prostate P. The longitudinal direction and direction of introduction of the device head 104 here correspond to the horizontal z direction. The B0 basic field is ideally aligned parallel to the z direction.

The probes described above are normally configured to be narrow so that they can be introduced easily through the body opening, and are then expanded to open up the MR coils contained therein (e.g. operating as antennas), and press them against the tissue to be examined. Expansion is generally brought about by pumping with air. Such probes are available, for example, from Siemens under the commercial name MEDRAD.

To reduce susceptibility problems at the air/tissue interface, fluids are also introduced in research applications. The introduction of fluids into a probe that is present inside a body is, however, considered problematical with respect to safety concerns, from a workflow point of view (complex handling) and also technically (due to the interaction of the fluid with electronic equipment).

FIG. 4 shows a sectional side view of an outline of a device head, in this instance in the form of the device head 104, of a possible probe, in particular the endorectal probe 103. The device head 104 (or endocoil) has a support rod 105 aligned in the z direction, in which the B0 main field is also aligned. Fastened to the support rod 105 are two MR coils 106 and 107, which are enclosed by a sheath 108 (made of flexible plastic for example), which can be inflated with air L. In the inflated state the sheath 108 has an ellipsoid or cylindrical basic shape for example. In the inflated state the MR coils 106 and 107 are also expanded, while when the sheath 108 is in the collapsed state they take up less space. To this end the MR coils 106 and 107 may be configured as flexible for example. In the inflated state the MR coils 106 and 107 are perpendicular to one another. They may serve as receive coils or receive antennas and output corresponding receive signals Rx1 and Rx2 for an MR spectroscopy examination. Inserted into the MR coils 106 and 107 here are lowpass filters in the form of capacitors C.

The fact that an object such as a probe (e.g. 103) present in a body K has a different susceptibility from the body K produces B0 differences precisely in the region of the organ to be imaged (e.g. a prostate P), which is generally close to an MR coil, the B0 differences possibly resulting in image quality problems.

A spherical structure (or in the broadest sense a structure similar to a sphere) with a different susceptibility from the surrounding tissue can be considered in a similar manner to a magnetic dipole. If the susceptibility (initially assumed to be constant) in the interior of the sphere is less than the susceptibility of the adjoining tissue, a field reduction results from the suppressing action of the more powerfully diamagnetic material of the sphere. It is also said that “the sphere suppresses the field”. If however the susceptibility in the interior of the sphere is greater than the susceptibility of the adjoining tissue, a field reduction is brought about by the suppressing action of the more powerfully diamagnetic tissue so “the sphere draws the field”. An inflated probe with a roughly spherical basic shape corresponds to the latter instance. The organ to be examined is frequently located in a position in relation to the probe, in which there is a relatively significant B0 field gradient, resulting in the problems set out above. The associated field disruption therefore corresponds to a so-called “magnetic dipole”.

DE 699 32 370 T2 discloses a localized magnetic field shim coil for correcting localized irregularities in a local region of the basic magnetic field in a magnetic resonance imaging system, that has multiple conducting elements, which are connected to a current source, the multiple conducting elements being adopted to be arranged adjacent to a localized region of an object to be imaged so that current flowing through the conducting elements generates a localized magnetic field that is essentially the same size as and counter to localized irregularities resulting in the basic magnetic field of the magnetic resonance imaging system, due to the geometric shape of the object and the magnetic susceptibility in the localized region to be imaged. A series-connected pair of a choke coil and resistor, or a series-connected pair of a resistor and capacitor/coil resonant circuit, is connected to each conducting element. The choke coils or capacitor/coil resonant circuits are dimensioned such that they suppress currents at frequencies that correspond essentially to the resonant frequency of the magnetic resonance system, and the resistors are dimensioned such that they ensure the symmetry of the current flowing through each conducting element.

DE 694 09 833 T2 discloses a magnetic resonance imaging apparatus for imaging a selected part of a patient's body, having a basic magnetic field generator and a gradient magnetic field generator. The basic field magnetic generator serves to generate a basic magnetic field, in which the induction flux of the basic magnetic field is fixed and expands in a fixed direction relative to the three orthogonal axes of a three-dimensional spatial reference system. It is possible to position the gradient magnetic field generator in the basic magnetic field in proximity to the body part to be imaged. A rail/coil arrangement has a moldable lining, which defines an inner zone, into which the body part to be imaged can be introduced for magnetic resonance imaging, the shape of the moldable lining being adjustable such that the rail/coil arrangement rests closely against the body part to be imaged. The rail/coil arrangement also has gradient magnetic coils that are arranged outside the moldable lining, enclosing it, to generate first, second and third magnetic gradient fields. The gradient magnetic coils are fastened to the moldable lining such that the first, second and third magnetic gradient fields are perpendicular to one another and the direction of the induction flux of the first, second and third magnetic gradient fields is stationary in relation to the rail/coil arrangement in order to essentially eliminate the relative movement between the magnetic gradient fields generated by the gradient magnet coils and the body part to be imaged, when the moldable lining positions the rail/coil arrangement closely against the body part. The rail/coil arrangement has a size and configuration such that the rail/coil arrangement can be placed in the basic magnetic field and can be positioned with any selected orientation in relation to the three mutually orthogonal axes of the three-dimensional spatial reference system.

### SUMMARY OF THE INVENTION

An object of the present invention is to overcome at least some of the disadvantages of the prior art and in particular to provide an arrangement that is particularly simple and economical to implement, as well as accurate, for performing a magnetic resonance measurement on a body.

The object is achieved by a device to be introduced at least partially into a body, the device having at least one MR coil that can be introduced into the body at the same time and at least one shim coil that can be introduced into the body at the same time.

The at least one shim coil serves to reduce the B0 inhomogeneities of the basic magnetic field as described above due to different susceptibilities between the device and the body (e.g. tissue and/or body fluid), by the at least one shim coil conducting electrical signals, in particular current signals, which generate a magnetic field (correction field), that compensates for the inhomogeneities (known as active shimming). The at least one shim coil may be operated by direct current.

The at least one shim coil may be configured so that its correction field in the body can be described using simple spherical surface functions, as interference fields, the cause of which is spatially removed from the body, also (at least approximately) have the form of such spherical surface functions of a low order therein—as described above. Such shim coils can be implemented simply and economically. Because they are arranged in particularly close proximity to a body part to be examined compared with a shim coil arranged outside the body, it is also possible to achieve particularly effective homogenization of the magnetic field.

The at least one MR coil is adopted to perform magnetic resonance tomography. To this end it may generate or read a magnetic field provided for magnetic resonance measurement. The at least one MR coil may be used as a transmit coil (TX), receiver coil (RX) or transmit/receiver coil (TX/RX). It may then also be referred to as an MRT or MRI coil. The use of MT coils is known in principle and does not need to be explained further hereto.

The device may in principle be any device that is suitable for introduction into a body. The device may be a medical device and may therefore be configured in particular for introduction into a human or animal body. It may also be a device for examining inanimate bodies, e.g. for examining objects in the field of archeology or for customs purposes.

The medical device may include or be a probe. This is typically removed from the body again after an examination. The probe may be an endoscopic probe, e.g. for examining the prostate or cervix. The device may include or be, for example, an endorectal probe, vaginal probes or a transesophageal probe. The device may then be an endoscope.

The medical device may also be an implant or the like. Such a device typically remains in the body for longer periods.

In an embodiment, the device has an expandable device head, in which the coils are housed. The expandable device head may serve in particular to press the device head against tissue at or in proximity to the region of the body to be examined.

A “device head” refers in particular to a region or segment of the device that is adopted for full introduction into the body. The device head can be a frontmost region of the device.

The capacity of the device head to expand means may be achieved by the device head having an expandable or expansible sheath. A sheathed volume of the external sheath can be enlarged. It may be possible to expand the sheath for example mechanically, pneumatically or hydraulically. The sheath may be an inflatable sheath. It may in particular be a balloon-type sheath. In the expanded state it may have a spherical, ellipsoid or hollow cylindrical basic shape. It may taper at one of its tips (along an introduction direction).

The coils (MR coil(s) and shim coil(s)) can be housed in the sheath. However other components of the device can also be housed in the sheath, e.g. electrical and/or electronic components such as capacitors, resistors, integrated circuits, etc. For example the at least one coil housed in the sheath may form a resonant circuit together with at least one capacitor.

In a further embodiment the device head has a support rod, which proceeds through the expandable sheath. The support rod holds the expandable sheath and may serve as a support element for components of the device housed in the sheath.

In a further embodiment, at least one shim coil is arranged in a fixed manner on or in relation to the support rod. This allows a particularly compact shim coil to be provided, for example if at least one shim coil is fastened on the support rod. The configuration can be implemented in a particularly simple and compact manner, if at least one shim coil is wound on the support rod. The support rod may then also function as a coil core.

At least one shim coil may be configured as a solenoid or a solenoid-type coil. At least one shim coil may be configured as a Helmholtz coil. At least one shim coil may be configured as a free arrangement of conductors. Such types of coil may be used alone or in combination.

Generally expandable and non-expandable shim coils can be provided, in particular can also be combined.

In a configuration that is advantageous for the simple introduction of the device head at least one shim coil can be expanded together with the device head (and can be collapsed again correspondingly together with the device head). To this end, at least one shim coil may be flexible and/or foldable. This has the advantage that the shim coil occupies particularly little space when the device head is introduced and it can also be expanded reliably.

In an embodiment, at least one shim coil is connected to the sheath directly or indirectly by at least one connecting element. This has the advantage that the expanding or collapsing sheath expands (e.g. opens up) or collapses (e.g. compresses or folds up) the shim coil at the same time.

The at least one MR coil may also be expanded or collapsed together with the device head, in particular it may be configured in the same manner as a shim coil.

In an embodiment, the device is designed to adjust the correction field generated by the at least one shim coil in a specific manner by the degree of expansion of the device head. This is achieved by the shape of the shim coils being adjusted by a spatial configuration of the shape of the expanded device head, if they can be moved, in particular expanded, with the device head. For example in the case of an inflatable sheath a pressure in the sheath may be able to be varied in a specific manner in order to adjust the correction field. The fixing of a particularly suitable expansion, represented, for example, by an internal pressure, determined, for example, by a calibration measurement.

In a further embodiment the at least one shim coil has a number of shim coils that can be activated and energized individually or in groups. This allows even a correction field with a complex configuration to be implemented in a relatively simple manner. This advantage is enhanced by the shim coils being activated adaptively (individually or in groups). In other words, their energization can be varied correspondingly to adjust the correction field. This is particularly advantageous in respect of compensating for any positional inaccuracy of the device. The fixing of a suitable activation of the shim coils, e.g. of a current strength to be conducted through the shim coils, may be determined, for example, by a calibration measurement.

Group activation of a number of shim coils may be achieved, for example, by the shim coils of a group being connected electrically to one another, e.g. in series and/or parallel. All the shim coils may also be connected electrically to one another, and in particular then form a coil system.

A shim coil may have one or more windings. If there are a number of windings, they may also be able to be activated adaptively individually or in groups.

An electrical current to be applied to the at least one shim coil can be applied in a fixed manner or can be determined from a calibration measurement.

A current direction through the shim coil(s) can be changed, e.g. switched to positive or negative, for example as a function of an orientation of the shim coil relative to the B0 main field.

In another embodiment, at least one shim coil is at a distance of between one and ten millimeters from the nearest outer surface of the device, in particular its device head. This allows local B0 hotspots or B0 inhomogeneities in the environment of the device, in particular its device head, to be prevented particularly effectively.

In a further embodiment, at least one shim coil is connected to at least one lowpass filter, in particular an RF lowpass filter. This protects it from unwanted signal coupling, in particular from RF coupling.

### BRIEF DESCRIPTION OF THE DRAWINGS

FIG. 1 is a sectional side view of a medical device to be introduced at least partially into a body according to a first exemplary embodiment.

FIG. 2 is a sectional side view of a medical device to be introduced at least partially into a body according to a second exemplary embodiment.

FIG. 3 is a sectional side view of an outline of an MR layout for a prostate examination.

FIG. 4 is a sectional side view of an outline of a conventional device head.

### DESCRIPTION OF THE PREFERRED EMBODIMENTS

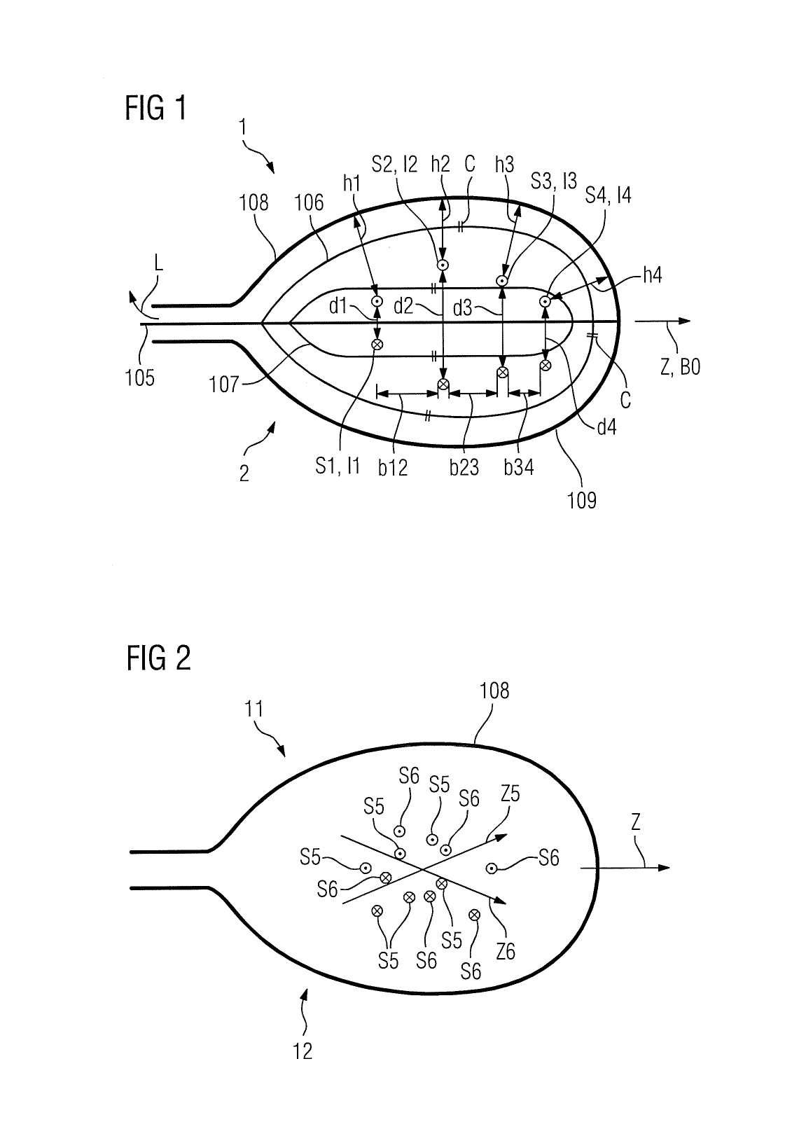

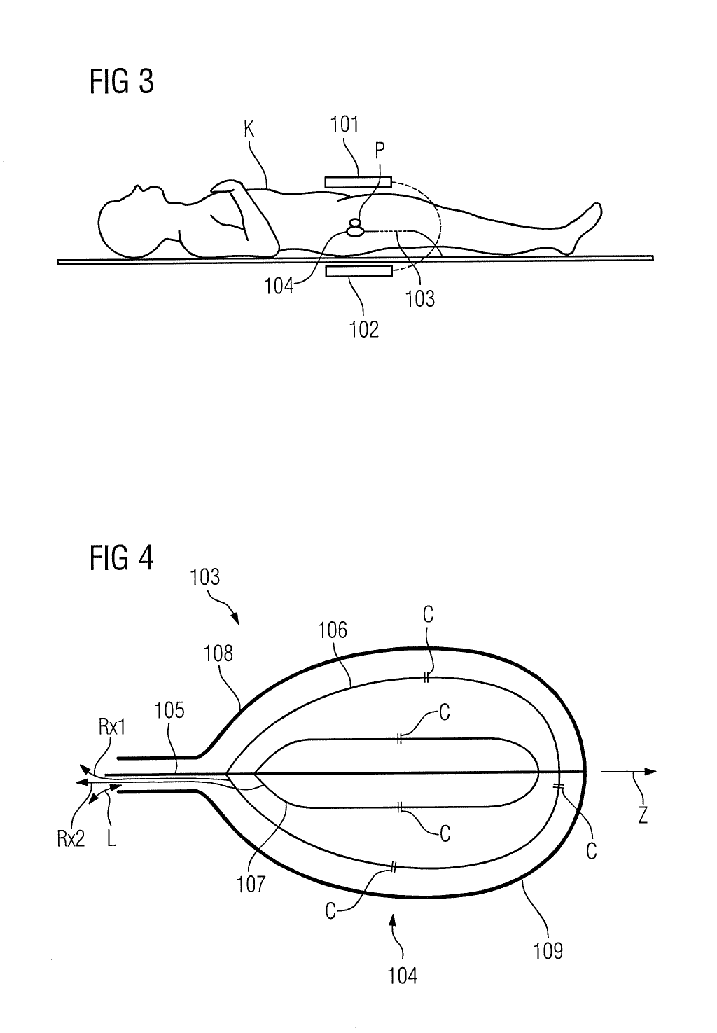

FIG. 1 shows a medical device in the form of a body probe 1 (e.g. an endoscope) with a device head 2. Like the device head 104 the device head 2 has a support rod 105, which is aligned in the z direction. The B0 basic field is also ideally aligned in the z direction. Also fastened to the support rod 105 here are two MR coils 106 and 107, which are enclosed by an expandable sheath 108. The sheath 108 may in particular be inflatable with air L, for example in the manner of a balloon. In its inflated state as shown it may be at least approximately spherical, ellipsoid or cylindrical in shape. In the inflated state the MR coils 106 and 107 are also expanded but when the sheath 108 is in the collapsed state they take up less space. The expansion of the MR coils 106 and 107 can be brought about for example by linking them to the sheath 108. To this end the MR coils 106 and 107 may be configured as flexible, in particular foldable. In the inflated state the MR coils 106 and 107 are also perpendicular to one another here, in particular when viewed along the z direction. Lowpass filters in the form of capacitors C are also inserted into the MR coils 106 and 107.

The MR coils 106 and 107 have MR coil terminals that allow connection of the MR coils 106 and 107 to a radio frequency (RF) transmitter or receiver, in order to cause the MR coils 106 and 107 to transmit RF pulses, or to receive MR signals, in the usual manner during the course of an MR examination of the patient in whom the body probe 1 has been inserted.

A number of shim coils S1, S2, S3 and S4 are also positioned on the device head 2 within the sheath 108. The shim coils S1 to S4 are arranged coiled in series along the z direction, specifically being wound around the support rod 105 here. The z axes of the shim coils S1, S2, S3 and S4, which lie in an x-y plane, are therefore parallel to the z alignment of the device head 2. This means that the shim coils S1 to S4 are arranged in a fixed manner on the support rod 105.

The diameters di (perpendicular to the z direction here) of at least two shim coils Si may generally be identical or different, with all the shim coils S1, S2, S3 and S4 here having different diameters d1 to d4.

The distances bij between two shim coils Si and Sj (along the z direction here) can also be identical or different.

The shim coils S1 to S4 individually can have one or more windings.

The shim coils S1 to S4 can be arranged in a fixed and close manner on the support rod 105, e.g. being wound around the support rod 105. They can also be expandable with the device head 2 or its sheath 108. To this end the shim coils S1 to S4 may be flexible and/or foldable and they may be connected directly or indirectly to the sheath 108. The expanding sheath 108 may then open up, e.g. unfold, the shim coils S1 to S4 at the same time. A collapsing sheath 108 can cause the shim coils S1 to S4 to collapse or fold up again in a similar manner.

The shim coils S1 to S4 can be supplied via terminals thereof with appropriate currents Ii so as to activate the coils S1 to S4 individually and adaptively. This may also include the situation of at least one of the shim coils S1 to S4 not being energized. These currents Ii can be direct currents. The shim coils S1 to S4 can in principle all be energized individually or may be connected electrically to one another in any manner, e.g. in series.

The shim coils S1 to S4 are also each at a distance hi of between one and ten millimeters from the nearest point on an outer surface 109 of the sheath 108.

Setting an internal pressure of the air in the sheath 108 and therefore a position of the shim coils S1 to S4 and/or variable energization of the shim coils S1 to S4 allow(s) the correction field generated by the shim coils S1 to S4 to be adjusted for a specific application in order to be able to produce a particularly informative MR image. This may be achieved, for example, by a calibration measurement.

FIG. 2 shows a medical device in the form of a body probe 11 (e.g. an endoscope) with a device head 12. The body probe 11 is similar to the body probe 1 in structure but the shim coils S5 and S6 are now configured differently. Only the sheath 108 and the two shim coils S5 and S6 of the body probe 11 are shown here. Both shim coils S5 and S6 have the same structure with identical diameter and three windings each.

Both shim coils S5 and S6 are now tilted in relation to one another and to the z alignment of the device head 12. This means that the z axes z5 and z6 of the shim coils S5 and S6 are at an angle to the z alignment of the device head 12, to the same degree here. These shim coils S5 and S6 can in particular be used advantageously to compensate for a positional tolerance, for example if the device head 12 is not aligned precisely parallel to the B0 basic field. To this end the shim coils S5 and S6 may in particular be energized differently. This may also include the instance wherein one of the two shim coils S5 and S6 is not energized.

Although the invention has been illustrated and described in detail using the exemplary embodiments shown, the invention is not restricted thereto and other variations can be derived therefrom by the person skilled in the art without departing from the scope of the invention.

Thus more or fewer than the illustrated MR coils and/or more or fewer than the illustrated shim coils can also be used in the device heads. The shim coils of both exemplary embodiments may also be arranged together in any manner, in other words shim coils aligned parallel to the z alignment of the device head and at an angle thereto can be used together.

I claim as my invention:

1. A device for introduction into the body of a patient, said device comprising:
a device body configured for at least partial intracorporeal insertion into an opening of the body of a patient;
at least one magnetic resonance (MR) coil carried by said device body for introduction into the body of the patient via said opening together with said device body;
said at least one MR coil comprising MR coil terminals adapted for connecting said at least one MR coil to a radio frequency (RF) component selected from the group consisting of an RF transmitter and an RF receiver, to transmit or receive RF signals in an MR examination of the patient;
at least one shim coil also carried by said device body for introduction into the body of the patient via said opening together with said device body and said at least one MR coil; and
said at least one shim coil comprising shim coil terminals adapted to connect said shim coil to a signal source that supplies said at least one shim coil with an electrical signal that causes said at least one shim coil to generate a magnetic field that compensates an inhomogeneity in a basic magnetic field generated during said MR examination.

2. A device as claimed in claim 1 wherein said device body comprises an expandable device head, in which said at least one MR coil and said at least one shim coil are contained.

3. A device as claimed in claim 2 wherein said device head comprises a support rod, and wherein said at least one shim coil is fixed on said support rod.

4. A device as claimed in claim 2 wherein said at least one shim coil is expanded together with said device head.

5. A device as claimed in claim 4 wherein said at least one shim coil is flexible or foldable.

6. A device as claimed in claim 1 comprising a plurality of shim coils having respective shim coil terminals allowing individual and adaptive supply of electrical signals respectively to the individual shim coils.

7. A device as claimed in claim 1 wherein said at least one shim coil is situated at a distance in a range between one and ten millimeters from a nearest outer surface of said device body.

8. A device as claimed in claim 1 comprising at least one lowpass filter electrically connected to said at least one shim coil.

9. A device as claimed in claim 1 wherein said device body is configured as an introcorporeal medical probe.

10. A device as claimed in claim 9 wherein said device body is configured as a probe selected from the group consisting of endorectal probes, vaginal probes, and transesophageal probes.

11. A device as claimed in claim 1, wherein said device body is configured as a medical implant.
